(19) United States
(12) Patent Application Publication
(10) Pub. No.: US 2002/0019031 A1
(43) Pub. Date: Feb. 14, 2002

(54) Chinese hamster ovary cells for producing proteins

(76) Inventors: Detlef Eisenkratzer, Penzberg (DE); Andre Dinter, Birrl (CH); Andre Kiesewetter, Lichtenberg (DE); Steffen Zeng, Kufstein (AT); Manfred Biselli, Julich (DE); Eric G. Berger, Schofflisdorf (CH)

Correspondence Address: KLAUS J. BACH & ASSOCIATES, PATENTS AND TRADEMARKS, 4407 TWIN OAKS DRIVE, MURRYSVILLE, PA, 15668, US

(21) Appl. No.: 09/835,089

(22) Filed: Apr. 13, 2001

Related U.S. Application Data
(63) Continuation-in-part of application No. PCT/EP99/07606, filed on Oct. 11, 1999.

(30) Foreign Application Priority Data
Oct. 14, 1998 (DE) 198 47 422.9

(51) Int. Cl. C12P 21/02; C12N 5/06

(52) U.S. Cl. 435/69.1; 435/358

(57) ABSTRACT

In Chinese hamster ovary cells for producing proteins, the growth phase is uncoupled from the production phase. In a method for preparing the Chinese hamster ovary cells and with the use of the Chinese hamster ovary cells for preparing proteins, a seed culture of high cell density is prepared which cells are grown to confluence and genetic material is transferred into the cells after the formation of a confluent surface (G1 phase).

This is a Continuation-In-Part application of international patent application PCT/EP/07606 filed Oct. 11, 1999 and claiming the priority of German application 198 47 422.9 filed Oct. 14, 1998.

### BACKGROUND OF THE INVENTION

The present invention relates to Chinese hamster ovary cells for producing proteins, to a method for preparing said Chinese hamster ovary cells and to the use of said Chinese hamster ovary cells for preparing proteins, preferably enzymes.

Using Chinese hamster ovary cells for producing proteins is known from the prior art. In this context, and by way of example, reference is made to Koplove H. M., Ann. Hematol. 1994; 68(3): 15-20. In the method described therein, the cells are contained in conventional stirred fermenters. Their use in immobilized form is known (Zeng S. et al., Biochem. Biophys. Res. Commun. 1997; 237 (3):653-658). However, there is a need for improving the yield of specific proteins in immobilized cells.

Accordingly, it is the object of the present invention to provide Chinese hamster ovary cells for producing proteins, which are suitable in immobilized form for use on an industrial scale.

### SUMMARY OF THE INVENTION

In Chinese hamster ovary cells for producing proteins, their growth phase is uncoupled from the production phase. A method is disclosed for preparing the Chinese hamster ovary cells and also the use of the Chinese hamster ovary cells for preparing proteins. For producing the Chinese hamster ovary cells a seed culture of high cell density is prepared which cells are grown to confluence and genetic material is transferred into the cells after the formation of a confluent surface (G1 phase).

The Chinese hamster ovary cells are preferably distinguished by a maximum secretion of the specific products in the G1 phase. G1 indicates a phase of the cell cycle in which the cell has a single deoxyribonucleic acid (DNA) content. Furthermore, in the cells the product formation rate preferably increases with decreasing growth rate, so that the maximum product formation rate is present at the minimum growth rate. According to the invention, the cell-specific product formation rate at minimum growth rate is at least as high as the specific product formation rate of conventionally transfected cells at high growth rate. Particular preference is given to an increase in the product formation rate by at least 50% for a drop in the S/G1 ratio by at least 50% below maximum. In other words, the decrease in the specific growth rate is inversely proportional to the increase in the cell-specific product formation rate. In this connection, S denotes the cell cycle phase in which deoxyribonucleic acid (DNA) is doubled, prior to the actual cell division.

The Chinese hamster ovary cells of the invention can be prepared by

a) preparing a seed culture of high cell density,

b) growing the cells to confluence in at least 24 h, and

c) transferring genetic material into said cells after the formation of a confluent surface.

According to the invention, preferably, a seed culture having a cell density of at least 1×10⁵ to 4×10⁵ cells/cm² is prepared. Highest preference is given to cell densities of from 2×10⁵ to 3×10⁵ cells/cm².

In this connection, a culture of adherent growing cells is denoted confluent when a free, unpopulated surface area is no longer present between the individual cells which are attached to a surface and the cells have formed a continuous layer on the surface. Preferably, the cells grow to confluence in 24 to 72 h. Particularly preferred are periods of from 30 to 60 h and most preferred are periods of from 40 to 50 h.

It is essential for the invention that genetic material is transferred after a continuous confluent area of cells has formed on the surface of the support and thus the cells are in the G1 phase of the cell cycle. In contrast to this, the previously known methods transfer genetic material into cells, which are in the S phase of the cell cycle, that is, after they have grown only to subconfluence.

Surprisingly, the effects according to the invention can also be achieved when using the cells of the invention in fluidized bed reactors despite the high shear forces occurring in these reactors. It is essential here that the genetic material is transferred after the cells have been arrested in the G1 phase of the cell cycle by immobilization on porous support material.

The transfer of the genetic material may be carried out in a manner known per se, for example by transformation or electroporation. The genetic material is preferably transferred by transfection of the cells.

Following the transfer of genetic material, suitable clones are selected. In other words, a screening is carried out in a manner known per se, in order to determine and cultivate the optimal cells, for example by sorting individual vital cells into a compartment of a tissue culture plate with the aid of a flow cytometer, subsequent testing of the productivity of the individual clones in an ELISA test and selecting the best clones by functional enzyme assays.

The method of the invention makes it possible to shift the peak of the recombinant gene product expression to the G1 phase. In contrast, conventional transfection methods lead to recombinant protein expression in the S phase of the cell cycle. Such cells have greatly reduced productivity when immobilized. On the other hand, the cells according to the invention are distinguished by a markedly higher productivity compared to that of conventionally transfected cells. In particular, the structural genes of the cells prepared in accordance with the invention have been found, surprisingly, to achieve an unexpected stability, which makes these cells suitable for production on an industrial scale.

The cells can be employed according to the invention in an immobilized form in fluidized bed reactors. In particular, the cells are suitable for producing various proteins, preferably enzymes. They may be used specifically for producing glycosyltransferases.

The invention is illustrated below in greater detail, on the basis of examples in comparison with the prior art (example No. 1).

### DESCRIPTION OF EXAMPLES

1. Conventional Transfection Method for Transferring Genetic Material (Prior Art)

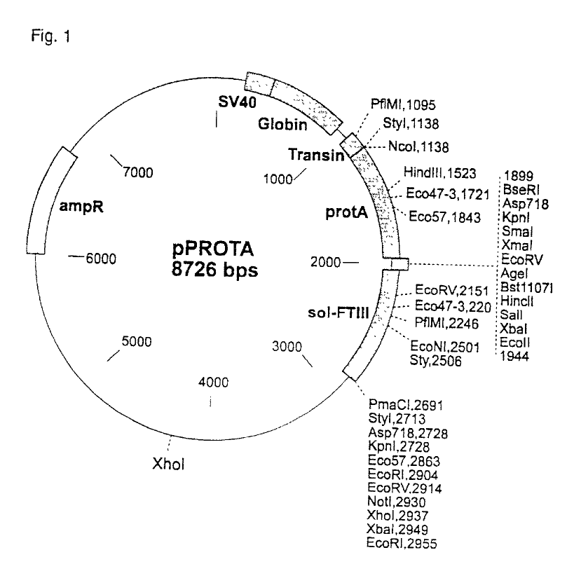

In the conventional transfection method, the Chinese hamster ovary K1 cell line, ATTC#CLR-9618, is cultured in round dishes in HAM's F12 medium from GibcoBRL Life Technologies, Gaithersburg, to 0.3×10⁶ cells per 3.5 cm² at 37° C. in a CO₂ incubator. After 24 hours, the cells have grown to subconfluence. For transfection, the cells are washed once in 2 ml of serum-free medium, HAM's F12 medium at a density of 2-3×10⁶ cells per 35-mm tissue culture dish. In sterile reaction vessels, 10 μl of Lipofectamine reagent from GibcoBRL Life Technologies, Gaithersburg and, respectively, 3 μg of recombinant plasmid DNA (pProtA/sol-FTIII, FIG. 1) coding for a glycosyl-transferase, and also 1 μg of plasmid DNA (pSV2neo) from Clontech, Palo Alto, USA, which as a selection marker codes for resistance to the antibiotic geneticin, are in each case dissolved in 100 μl of serum-free medium. The two solutions are combined, gently mixed and incubated at room temperature for 15 to 45 minutes to form the DNA-liposome complexes. These DNA-liposome complexes are then transferred into the cell suspension and gently mixed. After incubation at 37° C. in a CO₂ incubator for 5 hours, the cells are overlaid with 1 ml of HAM's F12 medium with 20% fetal calf serum (FCS) from Life Technologies, Gaithersburg. After 24 hours, the medium is replaced by 1 ml of HAM's F12 with 10% FCS and 800 μg/ml_{medium} G418 (=geneticin from GibcoBRL Life Technologies, Gaithersburg) to initiate selection. This medium is replaced with fresh medium of the same composition every 48 hours. From the sixth day after transfection onward, the latter medium is replaced with fresh medium of the same composition every 24 hours. On the 12^{th} day, individual clones are visible in the tissue culture dish, which are removed individually, using a Gilson ® pipette. The clones are separately cultured in the latter medium, which is replaced with fresh medium of the same composition every 24 hours. From the 18^{th} day after transfection, a functional enzyme assay is carried out to test the productivity of the individual clones. The precise experimental protocol is illustrated in more detail under point 3.

2. Method of the Invention for Transferring Genetic Material:

In the method of the invention, Chinese hamster ovary K1 cells from the strain collection ATCC#CLR-9618 are cultured in round dishes in HAM's F12 medium from GibcoBRL Life Technologies, Gaithersburg, to 0.6×10⁶ cells per 3.5 cm2 at 37° C. in a CO₂ incubator. After 48 hours, the cells have grown to confluence. In sterile reaction vessels, 10 μl of Lipofectamine reagent from GibcoBRL Life Technologies, Gaithersburg are diluted, and also 3 μg of recombinant plasmid DNA (pProtA/solFTIII, FIG. 1) coding for a glycosyltransferase and 1 μg of plasmid DNA (pSV2neo) from Clontech, Palo Alto, USA, which as a selection marker codes for resistance to the antibiotic geneticin, are in each case dissolved in 100 μl of serum-free medium HAM's F12 from GibcoBRL Life Technologies, Gaithersburg. The tow solutions are then combined, gently mixed and incubated at room temperature for 15 to 45 minutes to form DNA-liposome complexes. This is followed by removal of the culture medium from the round dish but without detaching the cells from the surface. Then the DNA-liposome suspension is added to the adhering cells. After incubation at 37° C. in a CO₂ incubator for 5 hours, the cells are additionally overlaid with 1 ml of HAM's F12 medium with 20% fetal calf serum (FCS) from GibcoBRL Life Technologies, Gaithersburg. After 6 days, the medium is replaced by 1 ml of HAM's F12 with 10% FCS and 800 μg/ml_{medium} (=geneticin from GibcoBRL Life Technologies, Gaithersburg) to initiate selection. This medium is replaced with fresh medium of the same composition every 24 hours. From the 15^{th} day after transferring the genetic material into the Chinese hamster ovary cells, individual clones are visible in the tissue culture dish. Using 1 ml of a ready-to-use trypsin/EDTA (trypsin/ethylenediaminetetraacetic acid) solution from GibcoBRL Life Technologies, Gaithersburg, all of the clones grown are rinsed off and are cultured again, in each case in a round dish with 1 ml of HAM's F12 medium with 10% FCS and 800 μg/ml_{medium} G418. After 3 days, the vital cells are isolaed by so-called single-cell sorting in the scattered light of a flow cytometer (e.g. FACScan® from Becton Dickinson and company, Franklin Lakes, USA). One cell is sorted into each compartment of a tissue culture plate with 96 wells and then cultured in HAM's F12 medium with 10% FCS and 800 μg/μl_{medium} G418, the medium being replaced with fresh medium of the same composition every 24 hours. After a further 3 days, the medium is replaced with fresh medium of the same composition every 48 hours. On the 23^{rd} day after transferring the genetic material into the Chinese hamster ovary cells, an enzyme-coupled immunological assay (ELISA; enzyme-linked immunosorbent assay), described in detail by Zeng S. et al., Biochem. Biophys. Res. Commun. 1997, August 28; 237(3): 653-658, is carried out to test productivity and to select the best clones. This is followed by subjecting the clones selected in this way to a functional enzyme assay (see point 3) to test their productivity.

Comments on 1) and 2):

A detailed map of the vector used, pProtA/sol-FTIII which codes for the human alpha-1,3-fucosyltransferase without membrane anchor, is depicted in FIG. 1. The transmembrane domain was removed from the available fucosyltransferase-III structural gene (Vestweber D., Zentrum fur Molekularbiologie der Entzundung, Westfalische Wilhelms-Universitat, Munster, FRG) and the soluble form with amino acids 44-361 (accession#X5378) was integrated into the vector pProtA.

3.Functional Enzyme Test for Assay for Determining Transferaseactivity:

The principle of the test for transferase activity is based on the transfer of radio-labeled fucose from GDP-fucose to the acceptor N-acetyllactosamine (LacNac, Sigma-Aldrich Chemie GmbH, Deisenhofen) in an enzyme-catalyzed reaction. The free acceptor and unreacted substrate are separated from the radioactive product via a chromatography column, and the radio-activity is measured as a measure for the amount of enzyme present. Initially, the enzyme is concentrated by binding to IgG-Sepharose. For this purpose, 1.5 ml of cell-free supernatant are incubated with 40 μl of support material consisting of 50% IgC-Sepharose beads in 0.05% Tween 20/5 mM Tris-HCl pH 7.6 at 4° C. overnight. After washing the support beads, the beads are directly and quantitatively employed in the enzyme assay. The assay mixture has a total volume of 89 μl and includes, besides the 40 μl of the 50% strength IgG-Sepharose support material, additionally, 40 μl of a reaction mixture consisting of 40 mm cacodylate buffer pH 6.2, 5 mM LacNAc as acceptor, 0.1 mM GDP-fucose as donor, 1 μl of C-GDP-fucose (50,000 cpm), 10 mM MnCl₂, 5 mM ATP and 10 mM fucose. The assay mixture is incubated with gentle shaking at 37° C. for 2 to 3 hours and produces an incorporated radioactivity content of between 1000 and 10,000 cpm. The reaction is stopped by adding cold water, purified via SepPak cartridges (manufactured by Waters) and then used to determine the radioactivity. The enzyme activity is calculated as follows: enzyme activity [U/1]=(product radio-activity [cpm]/total radioactivity used [cpm])×(amount of substrate [μmol]/incubation time [min]/volume of supernatant used [1.5 ml]).

4. Determination of Product Formation Kinetics in Suspension Culture:

As a prerequisite for determining product formation kinetics of the cells, the Chinese hamster ovary cells are kept in continuous culture in T75 tissue culture bottles (Greiner: Frickhausen, FRG). From this continuous culture, 2-3×10⁷ viable cells are then removed as a seed culture. For this purpose, the old culture medium is first removed and 2.5 ml of trypsin-EDTA from GibcoBRL Life Technologies, Gaithersburg are introduced into each T bottle, shaken and, after 1 min, removed again from the bottles. The T bottles are incubated for 5 min in the incubator, the cells are then detached by tapping and are rinsed off the bottle wall with 5 ml of a 3:1 mixture of DMEM and HAM's F12 medium with supplements (GibcoBRL Life Technologies, Gaithersburg), which has been pre-warmed to 37° C. The cell density is determined using 0.1 ml of cell suspension from each bottle. After calculating the cell concentration, the appropriate volume containing 2-3×10⁷ cells is removed, transferred to a sterile spinner bottle (Techne; Cambridge, GB) and made up to 100 ml. This corresponds to a starting cell concentration of 2-3×10⁵ cells/ml. The cells are cultured in a 3:1 mixture of DMEM and HAM's F12, with an osmolarity in each case of 350 mosmol/kg. In addition, the medium contains supplements in the form of essential amino acids, trace elements, vitamins, 6 mg/1 insulin, 6 mg/l transferrin, 0.1952 mg/l linoleic acid, 0.04636 mg/l thiol acid and 1% (v/v) fetal calf serum and 5 mmol/l glutamine and 24 mmol/l glucose. The spinner bottles are incubated on a stirrer plate at 60 rpm, a CO₂ content of 5% and at 37° C. At least every 24 hours, a sample of 10 ml is removed in a sterile manner from the spinner bottle and the cells are centrifuged at 200 g for 10 min. The product concentration in the cell-free supernatant is determined by means of ELISA. The cell pellet is resuspended in 2 ml of fresh and preincubated medium (5% CO₂, 37° C.), transferred into a Falcon culture tube (Greiner; Frickhausen, FRG) and incubated in the incubator for 2 hours. The cells are then centrifuged at 200 g for 10 min and the product concentration in the cell-free supernatant is determined by means of ELISA. The cell-specific productivity is calculated using the equation:

$$Productivity = \frac{Total amount of secreted product}{Incubation Time \times total cell number during incubation}$$

The cell cycle distribution is determined by means of a flow cytometer. For this purpose, the cells are resuspended in 70% strength alcohol and stored for permeabilization of the cell membrane at −20° C. overnight. The cells are then separated by centrifuging and 1×10⁶ cells are resuspended in PBS buffer from GibcoBRL Life Technologies, Gaithersburg, which buffer additionally contains 400 U/ml RNase (Sigma; Deisenhofen FRG) and 50 μg/ml RNase (Sigma; Deisenhofen, FRG) and 50 μg/ml propidium iodide (Sigma; Deisenhofen, FRG). After incubating the cells in the dark at 4° C. for 30 minutes, the cells are measured directly by means of a FACScan® flow cytometer from Becton Dickinson and Company, Franklin Lakes, USA. The program used for analysis was Cell-Quest (Becton Dickinson and Company, Franklin Lakes USA). The cell cycle phases were determined by mathematically evaluating the histograms obtained using the ModFit® program (Becton Dickinson and Company, Franklin Lakes, USA).

5a. Results of the Standard Method:

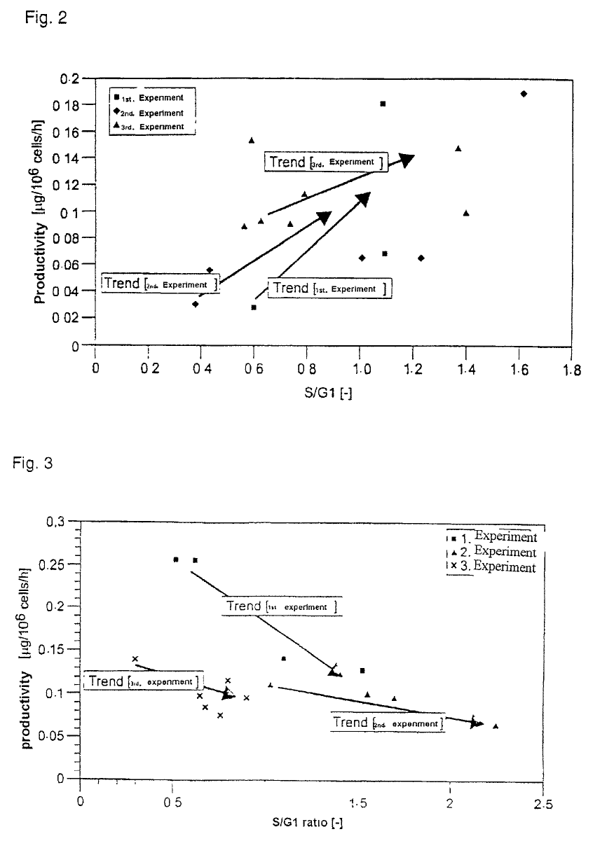

FIG. 2 depicts product secretion as a function of cell proliferation for three experiments in a suspension culture. The figure shows that product secretion increases with cell proliferation in the standard transfection method. During the phase of strong growth, product secretion is increased.

5b. Results of the Method of the Invention:

The characterization of product formation of cells into which genetic material was transferred by the method of the invention resulted in an opposite trend, as depicted in FIG. 3. With increasing proliferation, product secretion decreased in all experiments. Thus, when using the method of the invention, the maximum product formation rate is present at minimum proliferation. This fact is also of interest beyond pure productivity increase. Uncoupling of growth and product formation leads to significant improvements in the consumption rates. Since the cells produce well without growing, optimal utilization of the substrates employed is achievable.

Legends to FIGS. 1-3:

FIG. 1 shows a map of the vector used, pProtA/sol-FTIII

AmpR: ampicillin resistance for DNA production in E. coli,

SV40: SV40 virus transcription promoter

Globin: coding region for globin

Transin: coding region for transin as a signal sequence for transferase export

Sol-FTIII: structural gene of fucosyltransferase III without membrane anchor

ProtA: coding region for protein A to facilitate processing

Reference: Sanchez-Lopez R. et al., JBC 1998, 263(24): 11892-11899

FIG. 2 shows the results of the standard method

FIG. 3 shows the results of the method of the invention.

What is claimed is:

1. Chinese hamster ovary cells for producing proteins, said cells having a growth phase and production phase which are uncoupled from one another.

2. Chinese hamster ovary cells as claimed in claim 1, wherein said cells have a product formation rate which increases with decreasing specific growth rate.

3. Chinese hamster ovary cells as claimed in claim 1, wherein said ovary cells have maximum secretion of specific products in the G1 phase.

4. The Chinese hamster ovary cells as claimed in claims 1, wherein said cells have a maximum product formation rate at minimum specific growth rate.

5. The Chinese hamster ovary cells as claimed in claim 1, wherein a relatively low specific growth rate is coupled with a relatively high cell-specific product formation rate.

6. A method for preparing Chinese hamster ovary cells for producing proteins, said method comprising the steps of:
a) preparing a seed culture of said cells with high cell density,
b) growing the cells to confluence in at least 24 hours, and
c) after the formation of a confluent surface transferring genetic material to said cells.

7. The method as claimed in claim 6, wherein said seed culture has a cell density of at least 1×10⁵ cells/cm².

8. The method as claimed in claim 6, wherein said seed culture has a cell density of 1×10⁵ to 4×10⁵ cells/cm².

9. The method as claimed in any of claim 6, wherein said seed culture has a cell density of 2×10⁵ to 3×10⁵ cells/cm².

10. The method as claimed in any of claim 6, wherein said cells ore grown to confluence in 24 to 72 h.

11. The method as claimed in any of claim 6, which wherein said cells are grown to confluence in 30 to 60 h.

12. The method as claimed in claim 6, wherein said cells are grown to confluence in 40 to 50 h.

13. The method according to claim 6, wherein said Chinese hamster ovary cells are used in immobilized form.

14. The method according to claim 6, wherein said Chinese hamster ovary cells are used in fluidized bed reactors.

15. The use of the Chinese hamster ovary cells whose growth phase and production phase are uncoupled for producing proteins, preferably enzymes.
